(19) United States
(12) Patent Application Publication
(10) Pub. No.: US 2004/0161444 A1
(43) Pub. Date: Aug. 19, 2004

(54) Biocompatible hydrogel bone-like composites

(75) Inventors: Jie Song, Fremont, CA (US); Carolyn R. Bertozzi, Berkeley, CA (US); Eduardo Saiz, Berkeley, CA (US)

Correspondence Address: LAWRENCE BERKELEY NATIONAL LABORATORY, ONE CYCLOTRON ROAD, MAIL STOP 90B, UNIVERSITY OF CALIFORNIA, BERKELEY, CA, 94720, US

(73) Assignee: The Regents of the University of California, Oakland, CA

(21) Appl. No.: 10/740,739

(22) Filed: Dec. 18, 2003

Related U.S. Application Data
(60) Provisional application No. 60/434,596, filed on Dec. 18, 2002.

(51) Int. Cl. A61F 2/00

(52) U.S. Cl. 424/423

(57) ABSTRACT

A template-driven biomineralization process for making three-dimensional bonelike composites having direct and extensive mineral-substrate contact which provides high adhesion strength. The in situ generation of sufficient amounts of surface and interior carboxylates, through an increase in pH, serves as nuclear binding sites for mineral ions to promote high affinity 2-dimensional mineral growth at the substrate-mineral interface. The substrate for the bonelike composites is a hydrogel scaffold comprised of a polymerized base monomer having hydrolyzable ester side chains, crosslinked with a co-monomer and crosslinker. Hydrolysis of the ester containing side chains is preferably mediated by thermo-decomposition of urea.

### CROSS-REFERENCE TO RELATED APPLICATIONS

This application claims priority from U.S. Provisional Patent Application No. 60/434,596, filed on Dec. 18, 2002, which is hereby incorporated by reference in its entirety.

### STATEMENT OF GOVERNMENTAL SUPPORT

This invention was made during work supported by U.S. Department of Energy under Contract No. DE-AC03-76SF00098. The government has certain rights in this invention.

### BACKGROUND OF THE INVENTION

1. Field of the Invention

The present invention relates to the field of three-dimensional bonelike bulk composite materials obtained through the use of biocompatible hydrogel scaffolds and biomineralization.

2. Related Art

Bone is a complex tissue that serves many essential functions in the body. It protects organs, provides support and site of muscle attachment, generates blood cells and helps maintain essential ion levels. Structurally, natural bone is a composite of collagen, a protein-based hydrogel template, and inorganic dahilite (carbonated apatite) crystals. The unusual combination of a hard inorganic material and an underlying elastic hydrogel network endows native bone with unique mechanical properties, such as low stiffness, resistance to tensile and compressive forces and high fracture toughness. Throughout the cavities of bone, there are bone cells and a myriad of soluble factors and extracellular matrix components that are constantly involved with the bone formation and remodeling process. The unique biological functions and mechanical properties of bone are appealing to materials scientists, engineers and clinicians for a variety of applications, yet the complex nature of bone's structure has hindered real biomimetic design of artificial bonelike materials for a broad range of applications including the treatment of bone defects.

The formation of natural bone is thought to occur by directed mineralization of calcium apatite by highly acidic phosphoproteins attached to the collagen scaffold (Robey, P. G., Connective Tissue Research, 35(1-4): p. 131-6, 1996; Boskey, A. L., Connective Tissue Research, 35(1-4): p. 357-63, 1996.) Presumably, the acidic groups serve as binding sites for calcium ions and lower the interfacial energy between the organic matrix and the mineral phases (Weiner, S. and Addadi, L., Design strategies in mineralized biological materials. Journal of Materials Chemistry, 1997. 7(5): p. 689-702).

Recently, much progress has been made on self-assembling thin-film based template-directed biomineralization (Stupp, S. I. and Braun, P. V., Molecular manipulation of microstructures: Biomaterials, ceramics, and semiconductors. Science, 1997. 277: p. 1242-1248; Berman, A., Ahn, D. J., Lio, A., Salmeron, M., Reichert, A. and Charych, D., Total alignment of calcite at acidic polydiacetylene films—Cooperativity at the organic-inorganic interface. Science, 1995. 269(5223): p. 515-518; Xu, G. F., Aksay, I. A. and Groves, J. T., Continuous crystalline carbonate apatite thin films. A biomimetic approach. The Journal of American Chemical Society, 2001. 123: p. 2196-2203; Xu, G. F., Yao, N., Aksay, I. A. and Groves, J. T., Biomimetic synthesis of macroscopic-scale calcium carbonate thin films. Evidence for a multistep assembly process. Journal of the American Chemical Society, 1998. 120(46): p. 11977-11985), yet the application of such strategies is restricted to the surface coating of existing metal or alloy bone replacements. To simulate the unique mechanical and biological (dynamic resorption and tissue regeneration) properties of bone, one must mimic the complex composition and microstructures of bone in bulk materials rather than on coatings. So far, there has been no highly functional bulk bone-like composite material that meets all these requirements. Most attempts have been focused on polypeptides or polyelectrolytes based organoapatites prepared by co-precipitation of biominerals and polypeptides (Stupp, S. I. and Ciegler, G. W., Organoapatites: Materials for artificial bone. I. Synthesis and microstructure. Journal of Biomedical Materials Research, 1992. 26: p. 169-183; Stupp, S. I., Hanson, J. A., Eurell, J. A., Ciegler, G. W. and Johnson, A., Organoapatites: Materials for artificial bone. III. Biological testing. Journal of Biomedical Materials Research, 1993. 27: p. 301-311; Stupp, S. I., Mejicano, G. C. and Hanson, J. A., Organoapatites: Materials for artificial bone. II. Hardening reactions and properties. Journal of Biomedical Materials Research, 1993. 27: p. 289-299; Firouzi, A., Kumar, D., Bull, L. M., Besier, T., Sieger, P., Huo, Q., Walker, S. A., Zasadzinski, J. A., Glinka, C., Nicol, J., Margolese, D. I., Stucky, G. D. and B. F. Chmelka, Cooperative organization of inorganic-surfactant and biomimetic assemblies. Science, 1995. 267: p. 1138-1143). Although these organoapatites have shown some mechanical improvements over traditional bone implants, they are relatively expensive to make, structurally poorly characterized, and difficult to be further functionalized.

There has been considerable effort to mimic bone by the mineralization of polymer films with hydroxyapatite (Ca₁₀(PO₄)₆(OH)₂), the major inorganic component of natural bone. But overall, composite materials that integrate organic scaffolds and hydroxyapatite, and demonstrate the level of integration of natural bone, have not yet been achieved.

Hydrogel polymers are particularly appealing candidates for the design of highly functional tissue engineering scaffolds. The intrinsic elasticity and water retention ability of synthetic hydrogels resemble those of natural hydrogels, such as collagen matrices that are prevalent as structural scaffolds in various connective tissues including bone.

Poly(2-hydroxyethyl methacrylate), or pHEMA, is one of the most well-studied synthetic hydrogel polymers. With its high biocompatibility, pHEMA and its functionalized copolymers have become some of the most widely used synthetic hydrogels in tissue engineering. Applications include ophthalmic devices (e.g. contact lens) (Kidane, A et al., Biomaterials 1998, 19, 2051-2055), cartilage replacements (Oxley, H. R. et al., Biomaterials 1993, 14, 1064-1072), bonding agents in dental resins and bone cements (Yang, J. M. et al., Biomed. Mater. Res. 1996, 33, 83-88; Prati, C. et al., Clin. Mater. 1991, 8, 137-143), and various drug delivery vehicles (Lu, S.; Anseth, K. S. J. Controlled Release 1999, 57, 291-300; Sefton, M. V. et al., J. Controlled Release 2000, 65, 173-186). The present invention contemplates variations in the structures of the exemplified HEMA and HEMA derivatives according to variations of HEMA known in the art and as taught herein.

Calcium apatites are known to promote bone apposition and differentiation of mesenchymal cells to osteoblasts (Darimont, G. L. et al., Biomaterials 2002, 23, 2569-2575). Hydroxyapatite has limited solubility in water at neutral and basic pH but is highly soluble at acidic pH. Based on this property, a urea-mediated solution precipitation technique has been used in the preparation of composite ceramic powders (Blendell, J. E. et al., High Purity Alumina by Controlled Precipitation from Aluminum Sulfate Solutions. Am. Ceram. Soc. Bull. 1984, 63, 797-801 and De Jonghe, L. C. & He, Y., Composite Powder Synthesis. In: Ceramic Microstructures: Control at the Atomic Level, ed. Tomsia, A. P. & Glaeser, A. Plenum Press, New York, 1998, pp. 559-565). Wang et al, in Biomaterials 23: 4787-4791 (2002), describe a method of making biomimetic composites with polyamide(polyhexamethylene adipamide) by an ammonium phosphate solution stirred and heated to 70° C., with the pH kept between 10 and 12, to precipitate hydroxyapatite and uniformly disperse in the polyamide matrix.

To mimic natural bone synthesis in the fabrication of bone-like composite materials, one could start with a highly ordered molecular template to promote template-driven mineralization of crystalline apatites (Kim, H. M. et al., Calcif Tissue Int. 1996, 59, 58-63). Indeed, this approach has been pursued in the context of nanotube composites, using supramolecular aggregates of peptide-amphiphiles bearing biomimetically designed mineral nucleating ligands. Alternatively, one could start with composite materials with properly adhered amorphous or nanocrystalline osteophilic mineral compositions and encourage nature's remodeling pathway to eventually take over and engineer the initial mineral phase.

### BRIEF SUMMARY OF THE INVENTION

The present invention provides a bonelike composite, comprising: a hydrogel polymer scaffold having ester-containing side chains; an initial mineral deposition on the surface and the interior of the hydrogel polymer scaffold via contact with a mineralization mixture after partial hydrolysis of the ester side chains; and an extended mineral layer grown from the initial mineral deposition.

In one aspect of the invention, the hydrogel polymer scaffold has a water content between 20% and 100%, preferably about 40%.

The hydrogel polymer scaffold comprises a polymer having the structure shown in STRUCTURE 1, infra, —CH₂—CR²—COOR¹)ₙ— wherein R¹ is H or lower alkyl, R² is H or lower alkyl and n is preferably 10 to 100,000. In a preferred embodiment the polymer is pHEMA. In a preferred embodiment, R¹ is ethyl and R² is H and n is 10 to 100,000.

The polymer scaffold can further comprise 0.1% to 50% of a crosslinker, more preferably 2% to 10%. The crosslinker is preferably a compound of STRUCTURE II, infra, R³—C(CH₂)—CO—X—R⁴—X—C(O)—C(CH₂)—R^{3′}, wherein R³ and R^{3′} can be H or a lower alkyl, wherein the number of carbon atoms is preferably less than 10; R⁴ is [—(CH₂)ₙ—Y[—(CH₂)ₙ—]ₘ, wherein n and n′ can be independently from 1 to 10 carbon atoms and m=1 to 500,000. Y can be absent or O, S or NH and X is a heteroatom of O, S or N.

Furthermore, the crosslinker is preferably selected from the group consisting of diacrylates, diacrylamides, dimethacrylates or dimethacrylamides. In preferred embodiments, the crosslinker is ethylene glycol dimethacrylate, ethylene glycol dimethacrylamide, or a compound of STRUCTURE II, wherein R³is CH₃, R⁴ is CH₂CH₃ and X is O or N.

The polymer scaffold can further comprise 0.0% to 50% methacrylate co-monomers or methacrylamide co-monomers, preferably 0-25%. The co-monomer may bear functional groups including, but not limited to, anionic groups, polar ligands, aldehydes, ketones, phosphates, nucleic acids, amino acids, modified or phosphorylated or glycosylated or sulfated amino acids, peptides or proteins, carbohydrates, extracellular matrix components such as collagens and laminins, biodegradable motifs and polyethylene glycols.

In another aspect, the initial mineral deposition is a nanocrystalline or amorphous mineral deposit. The initial mineral deposition is comprised of a mineralization mixture comprised of inorganic components such as Ca²⁺, PO₄³⁻, OH⁻, CO₃²⁻, Cl⁻ and other inorganic elements. It is preferred that the ratio of Ca²⁺ to PO₄³⁻ ions is between 0.5 and 4, more preferably between 1 and 2. The mineralization mixture can be such mineral mixtures as hydroxyapatite (Ca₁₀(PO₄)₆(OH)₂), calcium carbonate, dicalcium phosphate, tricalcium phosphate, octacalcium phosphate, calcium phosphates having a stoichiometry that ranges from CaO-2P₂O₅ to 4CaO—P₂O₅, and solubility behavior, under acidic and basic conditions, similar to that of hydroxyapatite.

In another aspect of the invention, the extended mineral layer of the composite is about 1 to 7 μm in thickness. The extended mineral layer can be grown by extending the mineralization time and conditions. The present composite is then preferably attached to a bone in a vertebrate subject, or attached to an implant or organic-inorganic hybrid materials.

The present invention further provides a method for preparing a bonelike composite, comprising: (a) forming a hydrogel scaffold comprised of a crosslinked polymer having ester-containing side chains, (b) hydrolyzing a percentage of the ester side chains to form reactive acidic groups on the surface and the interior of the hydrogel; and (c) contacting the reactive acidic groups with a mineral to form a nanocrystalline or amorphous mineral deposit on said acidic surface and interior of the hydrogel.

In the method of the present invention, the hydrolysis of the ester side chains is catalyzed by the gradual addition or in situ generation of a composition that will thermally or aqueously degrade to release acid or base in a mild fashion in the interior and on the surface of the hydrogel. The acid used for hydrolysis is preferably selected from the group consisting of hydrochloric acid, sulfuric acid, phosphoric acid, acetic acid, formic acid, citric acid, carboxylic acid, other organic or inorganic acids miscible with water, and anything that could lead to the generation of these acids. The base is preferably ammonia, ammonium hydroxide, potassium or sodium carbonate, potassium or sodium bicarbonate, piperidine, imidazole, pyridine, other inorganic or organic bases, urea, and anything that could lead to the generation of these bases. An esterase could also be used to perform this function, i.e. cleavage of the R¹ ester.

In the present method, hydrolysis is preferably done by gradually increasing pH across the hydrogel scaffold through the addition or in situ generation of ammonia which can be generated by thermo-decomposition of urea through gradual heating of urea.

The pH is increased from about 1-3 to about 7-9 and the solution is preferably heated from room temperature to 95° C. at a heating rate between 0.1° C./min and 1° C./min, or more preferably a constant heating rate between 0.2 and 0.5° C./min. The thermo-decomposition of urea is preferably without agitation or stirring. It is further contemplated by the present method that heating of the solution is extended to about 10 to 12 hours to form an extended mineral layer upon the mineral deposit.

### BRIEF DESCRIPTION OF THE DRAWINGS

FIG. 1 is a qualitative depiction of the hydrogel scaffold in the mineralization process. FIG. 1A is a close-up depiction of the ester-containing side chains in a pHEMA hydrogel scaffold contacted with a mineralization solution. FIG. 1B is a close-up depiction of hydrogel scaffold after hydrolysis of the ester-containing side chains to create nucleation sites by pH increase. FIG. 1C is a depiction of the hydrogel scaffold during nucleation and two-dimensional outward growth of the initial mineralization layer and interior mineralization from the nucleation sites. FIG. 1D is a depiction of the hydrogel scaffold with an extended mineralization layer of micrometer thickness and extensive mineralization within the hydrogel.

FIG. 2 is a graph showing a qualitative depiction of urea-mediated, pH-dependent nucleation and growth behavior of a hydrogel scaffold (dotted curve) as it undergoes transformation from the hydrogel to a highly integrated composite in comparison to the solubility of Ca²⁺ (curve 1), the heterogeneous nucleation limit (curve 2) and the homogeneous nucleation limit (curve 3), as a function of pH.

FIG. 3 is a depiction of a strategy of making hydrogel networks bearing potential mineralization sites and functionalized groups by polymerizing monomers with ester side chains with crosslinkers and co-monomers bearing non-fouling and functional ligands.

FIG. 4 shows SEM micrographs and EDS and XRD analysis characterizing the extent of mineralization in a pHEMA-mineral composite. FIG. 4A is an SEM micrograph showing the side view of a cross-section of the pHEMA-mineral composite after extended mineralization. FIG. 4B is an SEM micrograph showing fully mineralized surface of pHEMA after extended mineralization,; the inset shows an expanded view of one spherical cluster of HA crystallites at a nucleation site. FIG. 4C is a calibrated EDS area analysis of the surface mineral layer shown in micrograph of FIG. 4B. FIG. 4D is the X-ray diffraction pattern of the resulting pHEMA-CP composite.

FIG. 5 is a SEM-associated EDS area analysis of a cross-section of the pHEMA-apatite composite, suggesting significant calcification throughout the hydrogel interior.

FIG. 6 shows SEM micrographs of the surface mineral patterns in a pHEMAm-mineral composite. FIG. 6A is a SEM micrograph of flower-like mineral patterns grown on the surface of pHEMAm hydrogel under the urea-mediated mineralization conditions. FIG. 6B is a SEM micrograph showing a mineral bundle deposited on the surface of the pHEMAm hydrogels.

FIG. 7 is the characterization of a pHEMA hydrogel mineralized with the relatively fast heating rate of 1.0° C./min. FIG. 7A is an EDS analysis performed over the composite which shows immediate damage of the surface under standard SEM operating condition, suggesting little mineral coverage on the hydrogel surface. FIG. 7B is an XRD analysis which shows the sporadic mineral deposits on the hydrogel surface that did not correspond to any crystalline calcium apatites.

FIG. 8 is the characterization of a pHEMA hydrogel mineralized with the moderate heating rate of 0.5° C./min. FIG. 8A is an EDS analysis performed on the composite, revealing a Ca/P ratio matching that of HA. FIG. 8B is an XRD analysis of the composite which suggesting a nanocrystalline or amorphous CP layer formed on the hydrogel surface.

FIG. 9 is the characterization of a pHEMA hydrogel mineralized with the moderate heating rate of 0.2° C./min. FIG. 9A is an EDS analysis performed on the spherical apatite aggregates formed on the hydrogel, confirming the expected Ca/P ratio. FIG. 9B is an XRD analysis performed on the composite material, confirming the formation of crystalline HA.

FIG. 10 shows the structures of monomers (101, 102) and co-monomers having non-fouling residues (103) and functional ligands (104, 105 and 106).

FIG. 11 is an EDS analysis on the mineralized pHEMA-co-5%-pGluMAm hydrogel. Similar results are obtained with mineralized pHEMA-co-5%-pGlyMAm, pHEMA-co-5%-pSerMAm hydrogels.

### DETAILED DESCRIPTION OF THE PREFERRED EMBODIMENT

### Definitions

The term “hydrogel” is used herein to refer to a porous three dimensional macromolecular network that swells in water and is comprised of one or more monomers polymerized with crosslinkers.

The term “crosslinker” is used herein in its conventional sense, i.e. a molecule that can form a three-dimensional network when reacted with the appropriate base monomers.

The term “crosslinked” is used herein to refer to when the crosslinker has reacted with the base monomer molecule to form a three-dimensional network.

The term “mineral” is used herein to refer to any inorganic compound, comprised of inorganic elements, including but not limited to, Ca²⁺, PO₄³⁻, OH⁻, CO₃²⁻, Cl⁻ and other trace inorganic elements. The inorganic compound can include, but are not limited to, such compounds as crystalline, nanocrystalline or amorphous hydroxyapatite (Ca₁₀(PO₄)₆(OH)₂), calcium carbonate, and calcium phosphates with solubility behavior, under acidic and basic conditions, similar to that of hydroxyapatite, including but not limited to dicalcium phosphate, tricalcium phosphate, octacalcium phosphate or calcium phosphates having a stoichiometry that ranges from CaO-2P₂O₅ to 4CaO—P₂O₅, with a definite composition and definite crystalline, nanocrystalline or amorphous structure.

The term “mineralization” is used herein to refer to integration of inorganic components onto or into a hydrogel scaffold.

The term “scaffold” refers to a three-dimensional porous polymeric structure with or without ionic sites or masked ionic sites along the polymer for mineral or other bone mineral attachment.

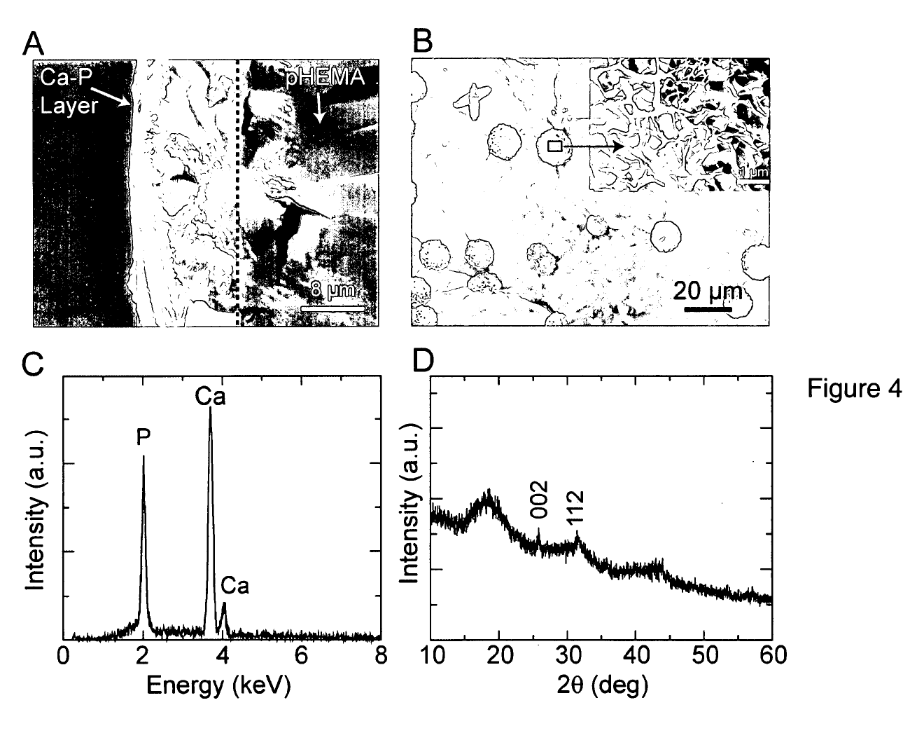

The term “nanocrystalline” is used herein to refer to a mineral formation that is not crystalline on a macroscopic scale and may be amorphous. It is illustrated in FIGS. 4-6 and FIG. 8.

The term “nucleation” is used herein to refer to the first step of mineralization where the inorganic cations are recruited to an anionic site in the hydrogel.

The term “lower alkyl” means straight-chain or branched saturated or unsaturated hydrocarbon residues with 1 to 20 carbon atoms, including substituted alkyl residues. A substituted alkyl residue is a straight chain alkyl, branched alkyl, or cycloalkyl group defined previously, independently substituted with 1 to 3 groups or substituents of halo, hydroxy, alkoxy, alkylthio, alkylsulfinyl, alkylsulfonyl, acyloxy, aryloxy, heteroaryloxy, amino optionally mono- or di-substituted with alkyl, aryl or heteroaryl groups, amidino, urea optionally substituted with alkyl, aryl, heteroaryl or heterocyclyl groups, aminosulfonyl optionally N-mono- or N,N-di-substituted with alkyl, aryl or heteroaryl groups, alkylsulfonylamino, arylsulfonylamino, heteroarylsulfonylamino, alkylcarbonylamino, arylcarbonylamino, heteroarylcarbonylamino, or the like.

The term “pHEMA” is used herein to refer to poly(2-hydroxyethyl methacrylate). The term “pHEMAm” is used herein to refer to poly(2-hydroxyethyl methacrylamide).

The term “CP” is used herein to refer to calcium phosphate.

The term “HA” is used herein to refer to hydroxyapatite.

The term “EDS” is used herein to refer to associated energy dispersive spectroscopy.

The term “XRD” is used herein to refer to X-ray diffraction.

The term “SEM” is used herein to refer scanning electron microscopy.

### Description of the Preferred Embodiment

To simulate the unique mechanical and biological properties of bone, such as high-affinity integration of mineral and organic phases, dynamic resorption and tissue regeneration, the general embodiment provides bone-like composites and microstructures in bulk materials rather than on coatings, which requires the design and fabrication of bulk polymeric scaffolds having a number of properties. In a general embodiment, these bulk polymer scaffolds should be robust and define the overall shape of the composite material and they should carry effective mineral binding sites (or “nucleation sites”) both within the interior and on the surface of the scaffold so that full penetration of minerals throughout the polymer network can be assured. It is preferred that the attachment of biological ligands that encourage cell attachment and spreading should be conveniently incorporated so that tissue integration will be promoted upon implantation of the composites into a subject vertebrate. Finally, the polymer scaffold should either be stable and long-lasting for long-term implantation application, or engineered to reach desired biodegradability for short-term applications.

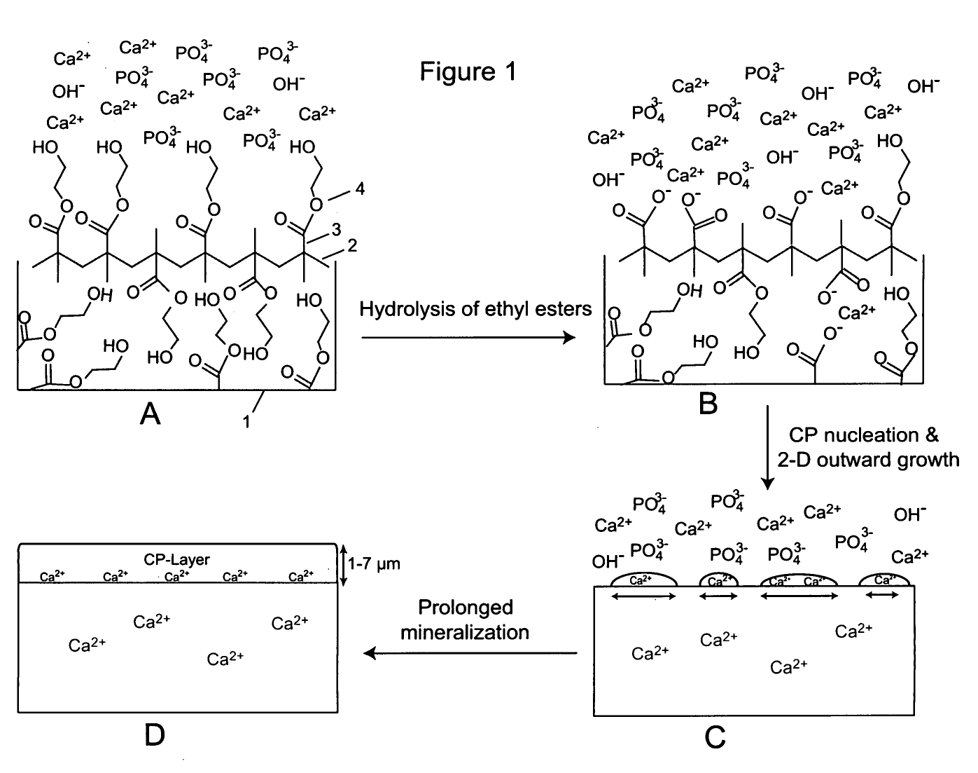

The composites are formed by a mineralization method that takes advantage of the dramatically different solubilities of minerals in acidic and basic aqueous media and the chemically labile nature of ester-containing side chains, which can be hydrolyzed to their corresponding carboxylic acid. Partial hydrolysis of the esters on the surface of and inside a hydrogel scaffold having ester-containing side chains forms reactive acidic (carboxylate) groups which act as nucleation sites. Referring now to FIG. 1, FIG. 1A represents diagrammatically a hydrogel 1 having a backbone 2 with a number of pendant side chains 3, both interior of the hydrogel and on the surface. The represented polymer is pHEMA. The side chains 3 contain an ester bond, as illustrated at 4. As shown in FIG. 1B, hydrolysis of the ester bonds by either acid, base or enzymes exposes reactive acidic groups. These reactive acidic groups can then be contacted with a mineral or a mineral-containing solution, which is illustrated as having ions Ca²⁺, PO₄³⁻, and OH⁻, to form a mineral deposit on the acidic surface of the hydrogel and an acidic interior in the hydrogel (FIG. 1B). Thus, as shown in FIG. 1C, heterogeneous nucleation from the nucleation sites (acidic groups) and high-affinity two-dimensional outward growth of a mineral, such as calcium apatite, occurs on the gel surface with extensive calcification inside the gel. Prolonged mineralization allows for the growth of a 1-7 nm thick mineral layer that covers the entire hydrogel surface (FIG. 1D).

Overall, this method provides a fast and convenient approach for producing robust mineralization of biocompatible composite materials with high quality interfacial integration between the mineral and the polymer substrate. The interfacial integration is described in detail below; the mineral deposit preferably grows 2-dimensionally initially at the gel-mineral interface to form either amorphous or nanocrystalline layer that robustly adheres to the hydrogel, rather than growth 3-dimensionally which results in the formation of large crystalline minerals that are easily detached from the hydrogel. Furthermore, this approach provides a foundation for integrating high-affinity template-driven biomineralization with the versatile properties of three-dimensional hydrogel scaffolds, and provides the basis for improved functionalized bone composites and replacements.

A. Hydrogel Polymer Scaffolds

In a typical hydrogel preparation, a hydrogel base monomer is combined with a co-monomer crosslinker, and water. Polymerization can be initiated through radical initiation. The well-mixed viscous solution should then be poured into a glass chamber and allowed to solidify. The gel should then be soaked in water for 2-3 days, with daily exchange of fresh water, to ensure the complete removal of unreacted monomers before being used for mineralization and further physical characterizations.

The hydrogel should be substantially comprised of a base polymer wherein the monomer is a substituted acrylate containing an ester side chain, having the general formula as shown in STRUCTURE 1 as follows:

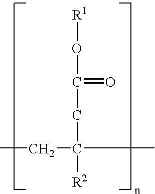

wherein R¹ can be H or lower alkyl, R² can be H or lower alkyl, and the number of the basic repeating units (n) can also vary, but is preferably 10 to 100,000.

In the exemplified preferred embodiment, the base polymer is poly(2-hydroxyethyl methacrylate) (pHEMA), where R¹ is —CH₂CH₃ and R₂ is H, and n is 10-100,000.

In other embodiments, the base monomer can be copolymerized with co-monomers which provide biomimetic properties and biocompatibility. These co-monomers should contain a polymerizable group, and should also preferably be methacrylates or methacrylamides. These co-monomers can bear anionic groups, or bear other types of functionalities such as polar ligands, nucleic acids, amino acids, modified or glycosylated amino acids, phosphorylated or sulfonated amino acids, peptides, proteins, and other functional groups, including but not limited to, polyethylene glycol (PEG), aldehydes, ketones, carbohydrates, extracellular matrix components such as collagens, laminins, biodegradable motifs such as phosphates, and other biological molecules. In preferred embodiments, the co-monomer crosslinker bears anionic sites that tend to bind to calcium ions, hydrophilic and non-fouling residues, and functionalities that mimic cellular adhesion peptides, including but not limited to peptides such as RGD, GRGDS and GRGD. Addition of these co-monomers, especially ones containing sequences known to contribute to the physical property of bone extracellular matrix, to the hydrogel scaffold should promote bone cell adhesion and proliferation over the composite surfaces. For example, Bab, et al., in U.S. Pat. No. 6,479,460, and Rodan, et al., in U.S. Pat. No. 5,461,034, disclose synthetic peptides, pseudopeptides, and pharmaceutical compositions having osteogenic activity which can be attached to the co-monomers as functional groups to make the biomimetic composites of the preferred embodiment, and are hereby incorporated by reference in their entirety. Different co-monomers may also be used to control porosity, the concentration of nucleation sites, and other properties.

The hydrogels in the preferred embodiment can have between 0.1-100%, preferably about 30-50% equilibrium water content (EWC). The EWC at room temperature is defined as the ratio of the weight of water absorbed by a dry hydrogel to the weight of the fully hydrated hydrogel. The amount of water absorbed by the hydrogel is determined from the weight of a freeze-dried gel (Wd) and the weight of the corresponding hydrated gel (Wh) according to the following equation:

EWC(%)=[(Wh−Wd)/Wh]×100

The desired EWC is obtained by hydrating the dry hydrogel with the desired amount of water.

Crosslinkers give support to the scaffold. In a preferred embodiment, the crosslinker is any di-acrylate or di-acrylamide, having the following general structure, STRUCTURE II:

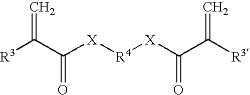

R³ and R^{3′} are identical or different and can be H or a lower alkyl, wherein the number of carbon atoms is from 1 to 10; R⁴ can be [—(CH₂)ₙ—Y—(CH₂)_{n′}—]ₘ wherein m is 1-500,000 and Y is O, S or NH or absent. n and n′ are independently from 1-10 carbon atoms; they are more preferably 1-4 carbon atoms. X is O, S or N. The length, m, of the crosslinker can be varied within the hydrogel as a method of controlling porosity, elasticity and mineralization.

The amount of crosslinker added to the base polymer is preferably 0.1% to 50% by weight, more preferably from 2% to 10% by weight, to afford hydrogels with varied degrees of crosslinking. The amount of crosslinking determines the overall porosity and strength of the hydrogel. A preferred crosslinker is ethylene glycol dimethacrylate, or STRUCTURE II, wherein R³ and R^{3′} are both CH₃, R⁴ is CH₂CH₂ and X is O.

Different crosslinkers may be used in accordance with the discussion above to provide biomimetic properties and biocompatibility. The various ligands, nucleic acids, peptides, and other functional ligands, discussed above for use in co-monomers, may be substituted for R₄ in STRUCTURE II.

B. Initial and Extended Mineral Layer

After hydrolysis of the esters of the base monomer, the acidic surface and interior of the hydrogel is contacted with a mineralization solution to form the initial mineral deposition via heterogeneous nucleation. Partial hydrolysis of the esters expose carboxylate groups which provide nucleation sites for the initial mineral deposition from the mineralization solution. This hydrolysis step is discussed below in connection with Section C.

The initial mineralization deposition is either nanocrystalline or amorphous. The crystals in the initial mineralization deposition are nano-sized, and produce no detectable signal by X-ray diffraction (XRD). Thus it is indistinguishable whether the initial mineral deposition is nanocrystalline or amorphous. No typical reflections corresponding to crystalline HA for the initial mineral deposit could be observed. Only a few broad reflections similar to those observed in hydrogels prior to mineralization are seen. This suggests that, although there is high affinity binding between calcium ions and the in-situ generated surface carboxylates, the spacing, order and/or alignment of these surface anionic ligands does not promote the epitaxial growth of crystallites, along any particular orientation, that are large enough to produce typical reflections of crystalline HA in XRD analysis. But the strong affinity between the mineral ion and the hydrogel carboxylates does lead to two-dimensional outward crystal growth of the initial mineral deposition layer from the individual nucleation sites.

The mineralization solution is preferably a mineralization mixture, comprised of such elements as Ca²⁺, PO₄³⁻, OH⁻, CO₃^{2—}, Cl⁻ and other trace inorganic elements such as crystalline, nanocrystalline or amorphous hydroxyapatite (Ca₁₀(PO₄)₆(OH)₂), calcium carbonate, and calcium phosphates with solubility behavior under acidic and basic conditions similar to that of hydroxyapatite, including but not limited to dicalcium phosphate, tricalcium phosphate, octacalcium phosphate or calcium phosphates having a stoichiometry that ranges from CaO-2P₂O₅ to 4CaO—P₂O₅.

The ratio of calcium to phosphate in the mineralization solution contacted with the hydrogel and in the subsequent mineral deposition, should be between 0.5 and 4, preferably from 1 to 2. For examples, the Ca/P ratio in the mineralization solution and the initial deposition would be 1 if dicalcium phosphate is used, 1.33 for octacalcium phosphate, 1.5 for tricalcium phosphate and 1.67 when hydroxy apatite is used.

Once the template surface is saturated with the initial nanocrystalline or amorphous mineralization deposition, the growth of the extended crystalline mineralization layer, which is composed of large platelet crystallites that are easily detectable by XRD, becomes energetically favorable. In a preferred embodiment, an extended mineral layer is grown from the initial mineral layer by prolonging the contact of the hydrogel with the mineral mixture until the extended mineral layer is a preferred thickness of about 1 to 7 μm.

C. Mineralization Method

As represented in FIG. 1 and discussed above, the formation of the robust mineralization layer on the hydrolyzed surface of the hydrogel is driven by the lower interfacial energy between the carboxylate rich gel surface and the mineral. An increase in pH hydrolyzes the ester side chain in the hydrogel to carboxylate groups. The exposed acidic carboxylate groups on the hydrogel surface and in the hydrogel interior act as nucleation sites and recruit Ca²⁺ ions. Two-dimensional outward growth from these nucleation sites create an initial mineralization layer on the surface and in the interior of the hydrogel, as more mineral is nucleated, grown and integrated. When the pH and temperature reach an equilibrium, the gel template surface will be fully covered with an initial nanocrystalline or amorphous mineralization layer. Calcium will also be internalized inside the hydrogel. The calcium ions recruit the mineral anions such as PO₄³⁻, HPO₄²⁻, H₂PO₄⁻ or OH⁻. The combined ions form a mineral deposit on and in the hydrogel scaffold. While calcium phosphate will be the major component of the mineral deposit, other minerals may be added.

In a dry state, the alkyl groups (R² of STRUCTURE I) would prefer to be exposed outside towards air, while the R¹ esters (which are less hydrophobic than the alkyl group R²) tend to extend into the hydrogel. However, once the esters are hydrolyzed into carboxylates, the anionic carboxylates prefer to be exposed to interact with the water environment. The esters on the base polymer ester side chains extend into or out of the hydrogel and can be oriented in any direction about the main carbon chain when soaked in water, since there is water both inside and outside the hydrogel. But steric hindrance, electrostatic repulsion, as well as more sufficient hydration with water are likely to lead to preferential exposure of surface carboxylates away from the inside of the hydrogel. Increased mineralization exposure increases the percentage of the esters hydrolyzed to carboxylate groups. In a preferred embodiment, only partial hydrolysis of all the R¹ esters in the hydrogel polymer scaffold is required. The percentage of esters hydrolyzed can be anywhere between 1% and 100% of the total ester side chains in the hydrogel scaffold, but preferably about 10-50%, producing a sufficient number of nucleation sites to create a strong mineral-gel interface such that the mineral-gel interface can withstand at least 5N loads without delamination as measured by the indentation test described infra.

Hydrolysis of the ester-containing side chains can be carried out by acid catalyzed hydrolysis, base catalyzed hydrolysis or through enzymatic hydrolysis by contacting the hydrogel scaffold with a composition or esterase that will thermally or aqueously degrade to release acid or base in a mild fashion. For example, one might consider bubbling gases such as NH₃ slowly into the mineralization media. Use of mild bases including, but not limited to, ammonium hydroxide, potassium or sodium carbonate, potassium or sodium bicarbonate, pipiradine, imidazole, pyridine, or anything that could lead to the generation of these bases, may also be adopted for use in the present process. Use of mild acids may be useful and in some embodiments may be preferred despite that generally, base catalyzed hydrolysis is more efficient than acid catalyzed hydrolysis. Appropriate mild acids useful for this process may include, but are not limited to, hydrochloric acid, sulfuric acid, phosphoric acid, acetic acid, formic acid, citric acid, carboxylic acid, other organic acids miscible with water, and any substance or compound that could lead to the generation of these acids.

It is preferred that the pH increase and hydrolysis of the ester-containing side chains be mediated by mildly basic compositions (pH 8-10) generated slowly in situ. The method of increasing the pH should allow a homogeneous variation of pH across the solution, avoiding any sudden local pH change that is commonly observed with strong base-induced homogeneous precipitation, thus ensuring hydrolysis of the ester side chains throughout the interior and on the surface of the hydrogel. Maintaining the final pH of the mineralization solution at around pH 8 also prevents competing homogeneous precipitation from the medium.

It is presently preferred that hydrolysis of the ester containing side chains be mediated by the thermo-decomposition of urea. Thermo-decomposition of urea allows a homogeneous variation of pH across the solution, avoiding any sudden local pH change and ensuring hydrolysis of the ester side chains throughout the interior and on the surface of the hydrogel.

Thermo-decomposition of urea in water produces ammonia, a mild base, which when contacted with the hydrogel, exposes reactive acidic groups (carboxylates) on the surface and interior of the hydrogel. The acidic groups on the interior of the hydrogel create a partially or substantially acidic interior, which has a high affinity for calcium ions, thus promoting extensive mineralization on the interior of the hydrogel.

The thermo-decomposition of urea in water which results in an increase in pH, resulting in the hydrolysis of surface esters and the precipitation of HA from the aqueous solution, is depicted by the following chemical equation:

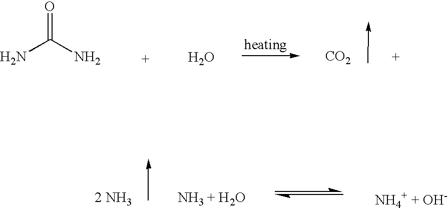

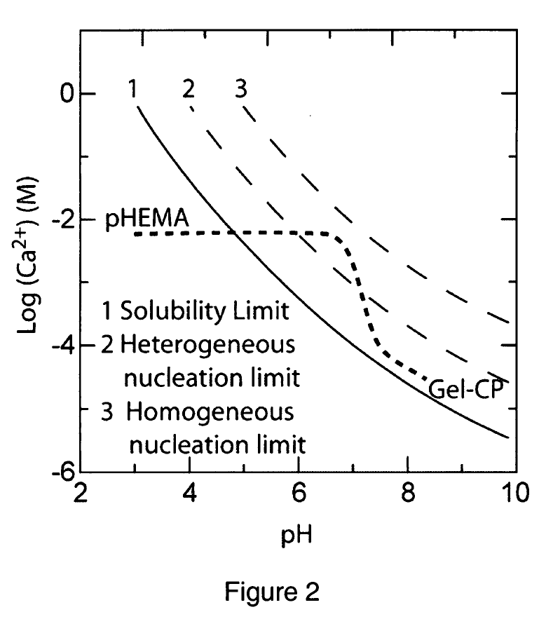

The mechanism of the presently preferred pH dependent mineral composite formation is further illustrated in FIG. 2. The dotted curve shows a qualitative depiction of urea-mediated, pH-dependent nucleation and growth behavior of a hydrogel scaffold as it undergoes the chemical and physical transformation from the hydrogel to a highly integrated composite. The solubility of Ca²⁺ is shown in curve 1, as a function of pH. It can be seen that increasing pH lowers Ca²⁺ solubility. Curves 2 and 3 represent qualitative depictions of typical nucleation behavior of calcium phosphate derived from basic nucleation theory. (See Blendell, J. E., Bowen, H. K. & Coble, R. L., High Purity Alumina by Controlled Precipitation from Aluminum Sulfate Solutions. Am. Ceram. Soc. Bull., 1984, 63, 797-801; De Jonghe, L. C. & He, Y., Composite Powder Synthesis. In: Ceramic Microstructures: Control at the Atomic Level, ed. Tomsia, A. P. & Glaeser, A. Plenum Press, New York, 1998, pp. 559-565).

The term “heterogeneous nucleation” refers to nucleation that occurs at the gel-mineral solution interface as the pH increases as the mineral ions nucleate onto the in situ generated carboxylate groups or other surface anionic groups and the mineralization layer is formed at the interface. “Homogeneous nucleation” begins to occur across the mineral solution at increased pH as the mineral precipitates out of solution. The present method avoids entering the homogenous nucleation region. At about pH 7-8, as the thermo-decomposition of urea occurs, the solubility of Ca²⁺ decreases, causing nucleation and calcification inside and on the surface of the hydrogel.

Therefore, it is presently preferred that the mineralization solution contain urea. The urea-mineralization solution can then be contacted with the hydrogel and heated for thermo-decomposition of the urea. Upon gradual heating of the urea-mineralization solution, without stirring from room temperature to 90-95° C. within 2 hours at a gradual heating rate of 0.1° C. to 1.0° C. per minute, urea will start to decompose and the pH will slowly increase to around pH 8. A lower heating rate and a longer overall mineralization time promotes the formation of better-merged CP layers on the gel surface. The longer exposure of the hydrogel to any given pH during the urea-mediated process is likely to lead to a more sufficient hydrolysis of the ethyl ester side chains, resulting in increased numbers of surface carboxylates that could serve as tight calcium ion binders and initial nucleation sites.

Constant stirring of the mineralization solution causes the mineral to homogeneously precipitate out of the mineral solution with the gradual increase of pH. No nucleation and high-affinity growth of calcium apatites on the hydrogel surface was observed with any tested heating rates under constant agitation. This suggests that direct stirring of the urea-mineralization solution interferes with the desired heterogeneous nucleation and mineralization growth on the in situ generated acidic gel surface and interior. It promotes undesirable homogeneous precipitation of the mineral across the solution, resulting in the formation of large amount of mineral precipitates.

The preferred time for a urea-mediated mineralization process that leads to the formation of a thin nanocrystalline or amorphous mineral deposition on the surface of the hydrogel is 1 to 12 hours, preferably 2-6 hours, with temperature rising from room temperature to 95° C. The heating rate can be a linear heating rate anywhere between 0.1° C./min and 1° C./min, preferably a constant heating rate anywhere between 0.2 and 0.5° C./min.

Alternatively, it is contemplated that in a specific embodiment, wherein enzyme digestion (e.g. proteases) is used to create the nucleation sites, then other side chains, such as amide linkages, may be suitable. Broadly speaking, the side chains may not need to be ester-linked so long as the heterogeneous nucleation at the nucleation sites is promoted in situ.

Once the template surface of the hydrogel is saturated with the initial nanocrystalline or amorphous mineralization deposition, the growth of the extended crystalline mineralization layer, which is composed of large platelet crystallites that are easily detectable by X-ray diffraction (XRD), becomes energetically favorable. By allowing mineralization to proceed for a longer time after reaching 95° C., mineral coatings with thicknesses up to several micrometers can be obtained, with good integration at the mineral-gel interface. The preferred time for a urea-mediated mineralization process to lead to the formation of thicker mineralization layers of about 1-7 μm thick on the surface of the hydrogel is at least about an additional 4+ hours after the mineralization solution has reached 95° C., preferably about another 12+ hours held at that temperature.

During the extended mineralization time, the growth of mineralization crystals forming three-dimensional aggregates on top of initial nucleation sites may be observed. The aggregates can be spherical with the centers being the nucleation site or of any other shape. The shape of three-dimensional crystal growth is of lesser importance to the current invention because it is likely that it will be remodeled by osteoclasts and osteoblasts once the composite has been implanted.

One feature of the composite made by this process is strong adhesion at the gel-mineral interface. The adhesion strength of the initial nanocrystalline or amorphous layer to the gel surface can be studied by microindentation analysis (Gomez-Vega, J. M. et al., J. Biomed. Mater. Res., 1999, 46, 549-559) performed on the surface of the mineral-hydrogel composite. No delamination of the mineral layer should be observed by SEM even after Vickers indentations with loads up to 15 N. In Example 3, neither the center nor the tip of the indenter markers with loads of 5N and 10N showed any signs of delamination of the CP layer on the pHEMA hydrogel, which is an indication of good adhesion at the mineral-gel interface. This represents a major improvement over current widely used simulated body fluid (SBF) mineralization methods, where most of the flake-like crystal apatite coatings obtained by mineralization in SBF on bioactive glasses, polymer scaffolds or collagen films are not robust and tend to delaminate easily upon drying.

D. Porosity and Functionalization of the Hydrogel

The porosity of the hydrogel scaffold may be adjusted by many available techniques including, solvent casting, particulate leaching, gas foaming and freeze drying (Misra, D. N. J. Dent. Res. 1985, 12, 1405-1408; Bradt, J.-H. et al., Chem. Mater. 1999, 11, 2694-2701; Liu, Q. et al., J. Biomed. Mater. Res. 1998, 40, 257-263; Murphy, W. L. et al., J. Am. Chem. Soc. 2002, 124, 1910-1917) and can be applied to this effort. The growth of mineral crystals inside the hydrogel scaffold is limited by both the space and the concentration of free anions achieved inside the already partially anionic hydrogel. Therefore an increase in porosity of the hydrogel may facilitate greater calcification as well as phosphate incorporation at the hydrogel interior. Such modifications could further enhance the degree of mineralization at the interior of the composite material and allow deeper tissue ingrowth.

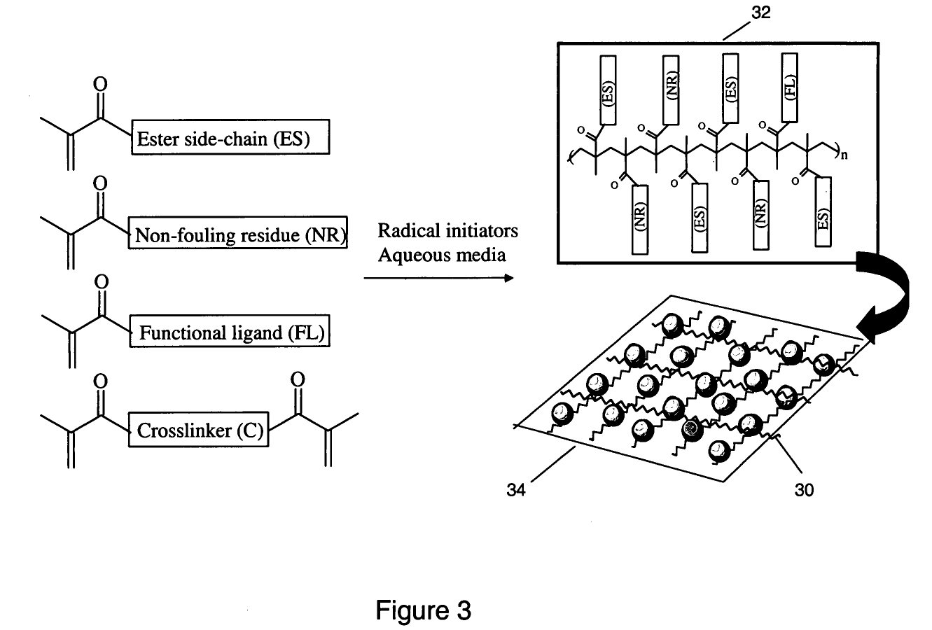

One way of controlling porosity of the hydrogel scaffold is by changing the crosslinker length and the percentage of crosslinker incorporation, which directly affects both the average pore size and the extent of crosslinking. Another way to increase porosity in the hydrogel scaffold is by incorporating various percentages of ionic co-monomers. Presumably, electrostatic repulsion between charged co-monomer sidechains would increase the pore size. The higher the percentage of ionic co-monomers incorporated, the stronger such repulsive interactions, and the higher the porosity of the hydrogel. However, highly ionic monomers at a high percentage may reduce the degree of crosslinking and length of the monomer chain. Shown in FIG. 3 is a general strategy for the synthesis of biomimetic polyacrylate-based hydrogel copolymers, wherein the base monomer is crosslinked with a crosslinker and co-monomers bearing various non-fouling residues and functional ligands on the left in FIG. 3A, to generate the hydrogel scaffold on the right in FIG. 3B. Porosity of the hydrogel will have a significant impact on not only the elasticity of the material, but also the extent of directed mineral growth and cell penetration. But high porosity may also contribute to low mechanical integrity of the resulting hydrogel scaffolds.

Referring now to FIG. 3, there is illustrated on the left a mixture of co-monomers having a variety of side chains (R¹ of STRUCTURE 1). Ester side chain (ES) is a simple cleavable side chain for generating a carboxyl mineralization site. Side chain NR is a non-fouling residue. Side chain FL is a functional ligand. Also illustrated is the representative crosslinker based on STRUCTURE 2. For co-monomers whose side chains that should survive hydrolysis (such as monomers bearing functional ligands, e.g. GluMAm, SerMAm, etc.), the amide linkage is intentionally designed so that when the monomers and co-monomers are copolymerized with a monomer and undergo hydrolysis, those amide-linked side chains can survive instead of being hydrolyzed.

This mixture of comonomers and crosslinker is added to an aqueous medium with standard free radical polymerization initiators to generate the structure 30, which is a hydrogel network bearing mineralization and functionalized domains. The network 30 is illustrated schematically at 32, showing that the various side chains are incorporated randomly according to the percentage of each component used. The predominant components will be the ES, with NR next most common, then FL. In a preferred application (discussed below), the network 30 will e attached to an implant or bone 34.

E. Applications

In addition to strong adhesion at the gel-mineral interface, the composites generated by this method have a nanocrystalline or amorphous mineralization layer with a structure and thickness ideal for bone implant applications. Analysis of calcium phosphate coatings on titanium implants has shown that resorption of the coating occurs mostly in the less organized apatite region and stops where the coating has higher crystallinity (Ratner, B. D. J. Mol. Recognit. 1996, 9, 617-625). Thus, the amorphous or nanocrystalline layer achieved by this method should promote resorption, bone integration, cell attachment and proliferation. In addition, earlier studies suggest that a thin layer of HA with thickness on the order of 1-7 μm provides a sufficient resorption timeframe to allow progressive bone contact with the implant substrate, and is therefore ideal for inducing integration of the material into natural bone (Ratner, B. D. J. Mol. Recognit. 1996, 9, 617-625). By contrast, classical plasma spray techniques applied to metal implants (Lu, S.; Anseth, K. S. J. Controlled Release 1999, 57, 291-300) produce HA coatings over 50 μm thick. The favorable properties of the hydrogel-calcium apatite composite obtained using the approach described herein should maximize the chance for initiating in vivo remodeling cascades and subsequent positive tissue-implant integration.

Thus, in practice it is contemplated that an implantable structure be formed in vitro according to a hydrogel adapted to fit a particular area of bony structure to be repaired or reconstructed. The composite is attached to bone in a vertebrate subject, or deposited on a hydrogel attached to an implant, or deposited on a hydrogel attached to another type of physiological implant. After mineralization according to the present procedures, the mineralized structure is implanted into the subject in the recipient site. Then, the implant is attached to the bony structure under physiological conditions, such as the modification or mediation of osteoclasts and osteoblasts. Such applications and extensions of the method would be known or understood by those skilled in the relevant art.

### EXAMPLE 1

### Preparation of Starting Materials—Preparing Base Monomer 2-Hydroxyethyl Methacrylate (HEMA) and Crosslinker

The crosslinker, ethylene glycol dimethacrylate (EGDMA), was used at 2%, 5% and 10% (by weight) to afford pHEMA gels with varied degrees of crosslinking. In EGDMA, R₃═CH₃, X═O, R₄═CH₂CH₂. These gels were found to have the same (40%) equilibrium water content (EWC). All subsequent experiments were carried out with gels crosslinked by 2% EGDMA.

2-Hydroxyethyl methacrylate (HEMA) was purchased from Aldrich and purified via distillation under reduced pressure prior to use. A standard radical polymerization protocol, as disclosed by Chilkoti, A., et al. (Analysis of polymer surfaces by SIMS. 16. Investigation of surface cross-linking in polymer gels of 2-hydroxyethyl methacrylate. Macromolecules, 1993, 26, 4825-4832) was applied for the preparation of pHEMA hydrogels crosslinked with EGDMA.

In a typical procedure, hydrogel preparation and polymerization is as follows. 500 mg of hydrogel monomer was combined with 10 μL of ethylene glycol dimethacrylate, 100 μL of Milli-Q water and 150 μL of ethylene glycol. To this mixture was added 50 μL each of an aqueous solution of sodium metabisulfite (150 mg/mL) and ammonium persulfate (400 mg/mL). Preferred radical initiators are sodium persulfate and ammonium persulfate. The solvent should be selected based on the ability to solubilize the monomers and crosslinkers used.

The well-mixed viscous solution was then poured into a glass chamber made by microscope slides and allowed to stand at room temperature overnight. The gels (5.5 cm×1.5 cm×1 mm) were then soaked in Milli-Q water for 2-3 days, with daily exchange of fresh water, to ensure the complete removal of unreacted monomers and radical initiators before they were used for mineralization and further physical characterizations. The thorough removal of these monomers and initiators from the formed hydrogel is also be important for biological applications as they may exert toxic or adverse effects in a biological environment.

### EXAMPLE 2

### Methods and Measurements Used to Characterize Hydrogels

Equilibrium water content (EWC) measurements. The EWC at room temperature is defined as the ratio of the weight of water absorbed by a dry hydrogel to the weight of the fully hydrated hydrogel. The amount of water absorbed by the hydrogel is determined from the weight of a freeze-dried gel (Wd) and the weight of the corresponding hydrated gel (Wh) according to the following equation:

EWC(%)=[(Wh−Wd)/Wh]×100.

Contact angle measurements. The contact angles of diiodomethane droplets against water on hydrogels were measured using the sessile drop method. A 3-5 μL droplet of diiodomethane was placed on the surface of a segment of hydrogel submerged in water. The static contact angles were measured with a goniometer from both sides of the droplet within ten seconds after depositing the drop, and the values were averaged. The contact angle was found to be 129° and 142° for the pre-hydrolyzed pHEMA and post-hydrolyzed pHEMA, respectively.

The hydrolysis of 2-hydroxyethyl esters during the thermo-decomposition of urea was expected to lead to an increase of surface hydrophilicity, which was confirmed by contact angle and EWC measurements. In a mock experiment, a segment of pHEMA gel was thermally treated as described above in a solution containing the same concentration of urea, without the presence of HA. Diiodomethane, a hydrophobic solvent that is known to from stable droplets on hydrophilic materials without noticeable penetration and contact angle hysteresis, was used for measuring contact angles against water on both the treated and untreated pHEMA gels. The contact angle of a diiodomethane droplet on the gel surface was found to increase from 129° to 142° upon the urea-mediated thermal treatment for two hours. The observed decrease in surface wettability by a hydrophobic solvent is consistent with the postulated in-situ generation of polar surface carboxylates during the urea-mediated mineralization. The hydrolysis also led to a slight increase (2-3%) in the EWC of the gel.

Structural Characterizations. Mineralized hydrogel strips were repeatedly washed in water to remove loosely attached minerals and soluble ions before they were freeze-dried for further structural analyses and mechanical characterization. The surface microstructures and crystallinity of the materials grown on the surface and inside of the hydrogel were analyzed by scanning electron microscopy (SEM) with associated energy dispersive spectroscopy (EDS) and X-ray diffraction (XRD).

SEM-EDS. All SEM micrographs of freeze-dried hydrogels and hydrogel-mineral composites were obtained with a ISI-DS 130C dual stage SEM with associated EDS. Samples were coated with either Au or Pt on a BAL-TEC, SCD 050 sputter coater to achieve optimal imaging results, or coated with carbon for EDS analysis. The imaging and analysis of composite materials were performed at 15 kV, and those of hydrogels were performed under reduced voltage (8-12 kV). The determination of Ca/P ratios of all composite materials were based on calibration using a standard synthetic HA sample.

XRD. The presence and overall orientation of crystalline phases in the precipitated mineral layers were evaluated by XRD with a Siemens D500 instrument using Cu Kα radiation.

Evaluation of mineral-hydrogel interfacial adhesion. Indentation test of adhesion. In order to evaluate the adherence of the mineral layers attached to pHEMA hydrogels, the relative crack resistance was qualitatively evaluated by indentation. The indentation test was performed using a Vickers indentor (Micromet, Buehler, Ltd., USA) that has a diamond pyramidal tip with a 136° angle between its faces. Loads from 5 to 15 Newtons were applied for 20 seconds for each measurement. After indentation, the samples where analyzed by SEM in order to check for delamination. Lack of delamination is an indication of strong adhesion between the mineral layer and the hydrogel substrate.

### EXAMPLE 3

### Mineralization of Hydrogels with the Urea-Mediated Process

A segment of pHEMA hydrogel was prepared according to Example 1 and soaked in an acidic solution (pH 2.5-3) of HA containing a high concentration of urea (2 M). HA (2.95 g) was first suspended into 200 mL of Milli-Q water with stirring, and 2 M HCl was added sequentially until all the HA suspension was dissolved at a final pH of 2.5-3. Urea (24 g) was then dissolved into the solution to reach a concentration of 2 M. Each hydrogel strip was then immersed into 50 mL of the acidic stock HA solution containing urea.

Upon gradual heating (without stirring) from room temperature to 90-95° C. (within 2 h) at a rate of 0.6° C. per minute, urea started to decompose and the pH slowly increased (around pH 8). Under these conditions, some hydrolysis of the 2-hydroxyethyl esters occurred at the hydrogel surface, promoting heterogeneous nucleation and 2-dimensional growth of calcium phosphate (CP). By two-dimensional growth, it is mean primarily crystal growth spreading along the surface of the hydrogel.

An SEM image of the pHEMA hydrogel showed 2-dimensional circular outward growth of calcium apatite from multiple nucleation sites on the acidic surface of the pHEMA hydrogel of Example 2 after 2 hours of heating time and mineral growth. The merging of circular mineral layers and the full coverage of the hydrogel surface with calcium apatite was also observed by SEM. The merge of circularly grown mineral layers resulted in sharp edges connecting individual CP patches.

The SEM-associated EDS area analysis of the mineral layer confirmed the chemical composition and Ca/P ratio (1.6±0.1) that is typical for HA. Synthetic HA was used to calibrate the determination of the Ca/P ratio. X-ray diffraction patterns of the pHEMA-mineral composite and unmineralized pHEMA gel showed no diffraction peaks corresponding to crystalline HA, which suggests that an amorphous or nanocrystalline mineral layer was formed on the pHEMA surface.

The mineral-hydrogel interfacial adhesion was tested by indentation analysis. The microindentation analysis was performed on the surface of the freeze-dried hydrogel-CP composite. An SEM showed an indent formed on the surface of mineralized pHEMA using a Vickers microindenter with a load of 5 N. However, the calcium phosphate layer showed no signs of delamination even after Vickers indentations with loads up to 15 N. Neither the center nor the tip of the indenter markers with loads of 5N or 10N showed any signs of delamination.

### EXAMPLE 4

### Extended Mineralization of pHEMA via a Urea-Mediated Process

By extending mineralization time 10-12 hours, mineral coatings with thicknesses up to several microns were obtained, with good integration at the mineral-gel interface as shown in a side view image of the composite (FIG. 4A). FIG. 4A is an SEM image of a section of a pHEMA hydrogel with an extended Calcium-Phosphate layer shown on the left. The dotted line indicates the mineral-gel interface. The sample stage was tilted at 45°. Note the micron scale thickness of the mineral layer and the fine integration at the mineral-gel interface.

Once the surface was fully covered with the amorphous apatite layer, the growth of HA crystals forming spherical aggregates on top of initial nucleation sites (centers of circular apatite rings) was observed (FIG. 4B). These mineral spheres are composed of plate-like crystallites, a typical morphology observed with the crystalline apatite grown on bioactive glasses polymer substrates or collagen films using SBF mineralization.

Referring to FIG. 4C, the results of EDS analysis performed on the spherical apatite aggregates confirmed the expected Ca/P ratio (1.6±0.1) for crystalline HA. SEM-associated EDS area analysis of a cross-section of pHEMA-apatite composite displayed a large intense peak corresponding to calcium, suggesting significant calcification throughout the hydrogel interior. In XRD performed on the composite material, the results as shown in FIG. 4D, typical reflection peaks corresponding to crystalline HA, at (002) and (112), were detected, suggesting the crystalline nature of the spherical HA grown on top of the initial calcium apatite nanocrystalline or amorphous layer. XRD also detected a preferential alignment along the c-axis. It is worth noting, however, that HA crystals formed by adventitious precipitation often adopt a similar preferential orientation. This suggests that the observation of a preferential alignment of the apatitic crystal lattice in a composite material does not necessarily reflect a specific interaction with the underlying substrate.

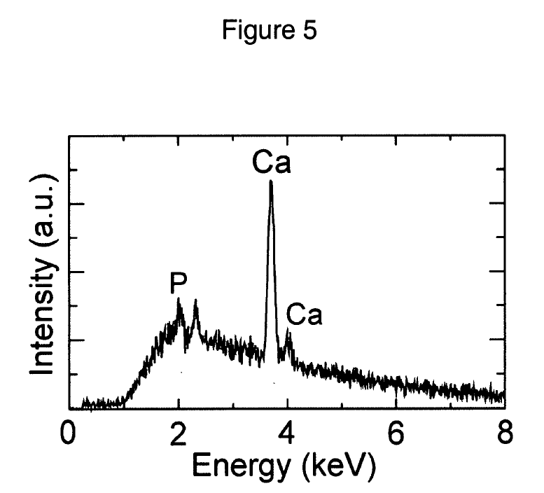

A cross-section examination of the composite material revealed that there were significant degrees of calcification inside the hydrogel as well, although the degree of phosphate incorporation was limited. The extensive calcification of the hydrogel interior may be promoted by the partial hydrolysis of the 2-hydroxyethyl ester side chains inside the pHEMA gel. The growth of calcium apatites inside the pHEMA scaffold is limited by both the space and the concentration of free anions achieved inside the already partially anionic hydrogel. SEM-associated EDS area analysis of the cross-section of pHEMA-apatite composite, shown in the micrograph of FIG. 5E, suggested significant calcification throughout the hydrogel interior. This is suggested by the intense peak of Ca at around 3.8 kV.

### EXAMPLE 5

### Undesirable Surface Mineral Patterns of pHEMAm Gels via a Urea-Mediated Process

To better understand the relationship between surface chemistry of the substrate and the mineralization pattern of the composite material, the same mineralization technique used in Example 3 was applied to poly(2-hydroxyethyl methacrylamide) (pHEMAm), a hydrogel that is not prone to side chain hydrolysis under the mineralization conditions. In this polymer, R₁═CH₂CH₃, R₂═H, C(O)O linkage is replaced with C(O)N, and n=10-100,000 (STRUCTURE 1).

A standard radical polymerization protocol was used for the preparation of pHEMAm as disclosed in Chilkoti, A., Lopez, G. P. & Ratner, B. D., Analysis of polymer surfaces by SIMS. 16. Investigation of surface cross-linking in polymer gels of 2-hydroxyethyl methacrylate. Macromolecules, 1993, 26, 4825-4832. 2-Hydroxyethyl methacrylamide (HEMAm) was synthesized through direct coupling of ethanolamine with methacryloyl chloride under slightly basic (pH 8) conditions. To 20 mL of ice-cold methanolic solution of ethanolamine (2 mL, 33 mmol) was slowly added 3.26 mL (34 mmol) of methacryloyl chloride (diluted in 20 mL of THF). Potassium hydroxide (1 M, aqueous) was added to maintain the solution pH at 8-9 throughout the reaction. The mixture was warmed to room temperature over 2 h and stirred for another 2 h before it was quenched by the addition of hydrochloric acid to a final pH of 5. The product was concentrated and redissolved in cold ethanol to precipitate the potassium chloride salt. After silica gel flash chromatography purification (chloroform: methanol/9:1), the product (R_{f} 0.5) was isolated in 95% yield. ¹H NMR (500 MHz, CD₃OD): δ 7.10 (1H, b), 5.55 (1H, s), 5.16 (1H, s), 3.51 (2H, t, J=5.5 Hz), 3.25 (2H, q, J=5.0 Hz), 1.76 (3H, s); ¹³C NMR (125 MHz, CD₃OD): δ 169.31, 139.06, 119.82, 60.71, 41.96, 18.09; HRMS FAB⁺ (NBA): C₆O₂NH₁₂ [M+H]⁺, calcd 130.0868, found 130.0868.

The crosslinker, ethylene glycol dimethacrylate (EGDMA), was used at 2% (by weight) to crosslink the pHEMAm gel.

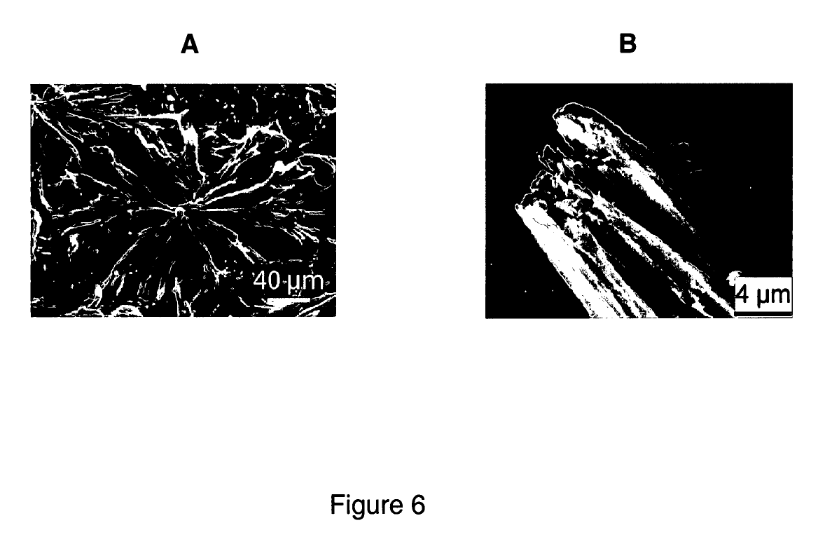

An entirely different surface mineral pattern was obtained with pHEMAm hydrogels when the urea-mediated mineralization method was applied. As shown in FIG. 6A, the pHEMAm hydrogel was patterned with flowerlike minerals, with much less extensive surface coverage even after 12 hours of mineralization. The apatite grown on pHEMAm was crystalline as suggested by both a dark field optical image and XRD of the composite material, with major reflections matching with those of crystalline hydroxyapatite (HA).

FIG. 6A shows an XRD analysis of the mineral pattern. The major reflections and relative intensities of an XRD analysis of the composite suggest a preferential alignment along (002), with the c-axis perpendicular to the substrate. SEM micrographs revealed further details of the mineral pattern, showing an upward growth of the bundles of whiskers away from the gel surface (FIG. 6B). This, along with the relatively low surface mineral coverage, is consistent with the decreased affinity between calcium apatite and the neutral hydrogel surface of pHEMAm.

An EDS analysis performed on the mineral bundles of the pHEMAm hydrogels again revealed a Ca/P ratio (1.6±0.1) matching HA. X-ray elemental mapping of Ca and P showed that the mineral patterns were composed of uniform calcium phosphate apatite. The dark hydrogel surface positions, devoid of flowerlike mineral patterns, were in agreement with the low-calcium binding nature of pHEMAm. Surface chemistry appears to play a role in determining both the extent and the pattern of the mineralization (2-D vs. 3-D mineral growth). Tightly bound HA on a pHEMA hydrogel prefers to spread on the surface, forming a circularly grown mineral layer that eventually covers the entire gel surface. By contrast, more loosely bound HA, as in the case of pHEMAm, grows more readily in three dimensions above the surface rather than integrating with the hydrogel. Therefore, use of pHEMAm as a base monomer is not preferred because there was no calcification or phosphate incorporation detected at the interior of the hydrogel and lower mineralization with the hydrogel.

### EXAMPLE 6

### Effect of Different Rates of Temperature Increase in the Urea-Mediated Process

Using a homemade heating system with fine temperature control, pHEMA hydrogels of Example 1 were mineralized using the same acidic HA-urea stock solution from room temperature to 95° C. with heating rates of 1.0, 0.5, 0.2 and 0.1° C./min, respectively. No agitation of the mineral stock solution was applied and the mineralization process of Example 3 was terminated once the temperature reached 95° C. The resulting composites were then examined for surface mineralization patterns via SEM, elemental compositions via EDS and crystallinity of the mineral components via XRD.

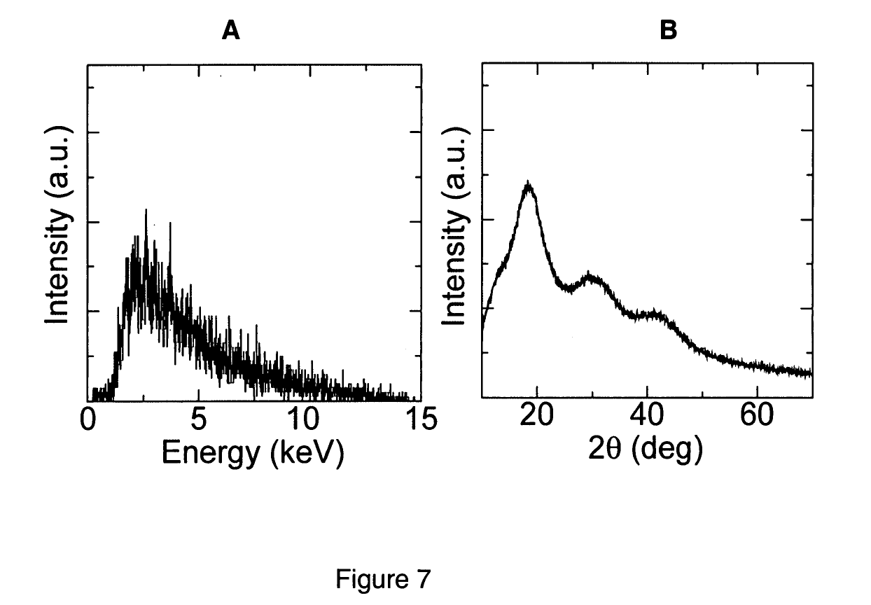

Referring now to FIG. 7, a relatively fast heating rate such as 1.0° C./min did not lead to a level of mineralization of the pHEMA gel that was detectable by either SEM or the associated EDS analysis. The EDS analysis performed over the composite led to immediate damage of the surface under standard SEM operating condition (15 kV) and the results are shown in the EDS analysis of FIG. 7A. Such high surface sensitivity to the electron beam is typical for unmineralized hydrogels. The random and featureless deposits on the hydrogel surface did not correspond to any crystalline calcium apatites as evidenced by the XRD analysis shown in the micrograph of FIG. 7B, which lacks characteristic reflections of calcium phosphates. Overall, these data suggests that a fast heating rate such as 1.0° C./min and insufficient mineralization time (a total of 70 min linear heating from room temperature to 95° C.) do not lead to adequate mineralization of the hydrogel using the urea-mediated protocol described above in Example 3.

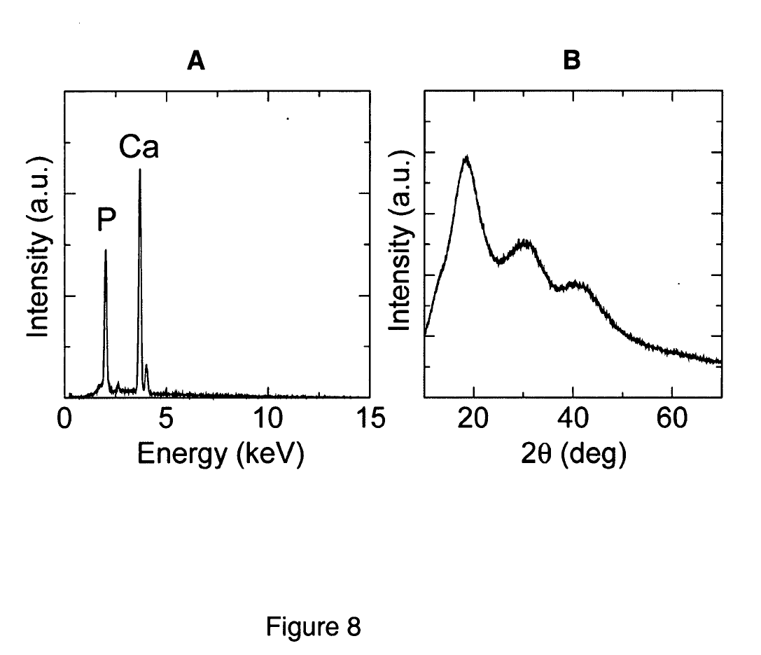

When a 0.5° C./min heating rate was applied, the formation of circular mineral layers on the hydrogel surface was observed, with similar 2-dimensional outward growth pattern formed around individual nucleation sites. X-ray elemental mapping of Ca and P showed that the circular mineral patterns were composed of uniform calcium phosphate. EDS analysis performed on the composite revealed a Ca/P ratio matching that of HA (FIG. 8A). XRD of the composite, however, showed no reflections matching with those of crystalline calcium apatites, suggesting that the circular CP layer grown on the hydrogel surface were either nanocrystalline or amorphous in nature (FIG. 8B).

When the linear heating rate (from room temperature to 95° C.) was lowered to 0.2° C./min, the surface of the hydrogel-mineral composite was fully covered with well-merged circular mineral layers. Scratching the surface mineral layer with a razor blade did not lead to any delamination, indicating good adhesion strength at the gel-mineral interface. In addition, the growth of HA crystals forming spherical aggregates on top of the initial nucleation sites (seen as centers of circular mineral rings) was observed. These mineral spheres are composed of plate-like crystallites, a typical morphology observed with the crystalline apatite grown on bioactive glasses, polymer substrates or collagen films using such methods as simulated body fluid (SBF) mineralization (See Kokubo, T., et al., Acta Mater., 1998, 46, 2519-2527; Rhee, S.-H. & Tanaka, J., Biomaterials, 1999, 20, 2155-2160; Murphy, W. L. & Mooney, D. J., J. Am. Chem. Soc., 2002, 124, 1910-1917 ; Saiz, E., et al., Biomaterials, 2002, 23, 3749-3756).

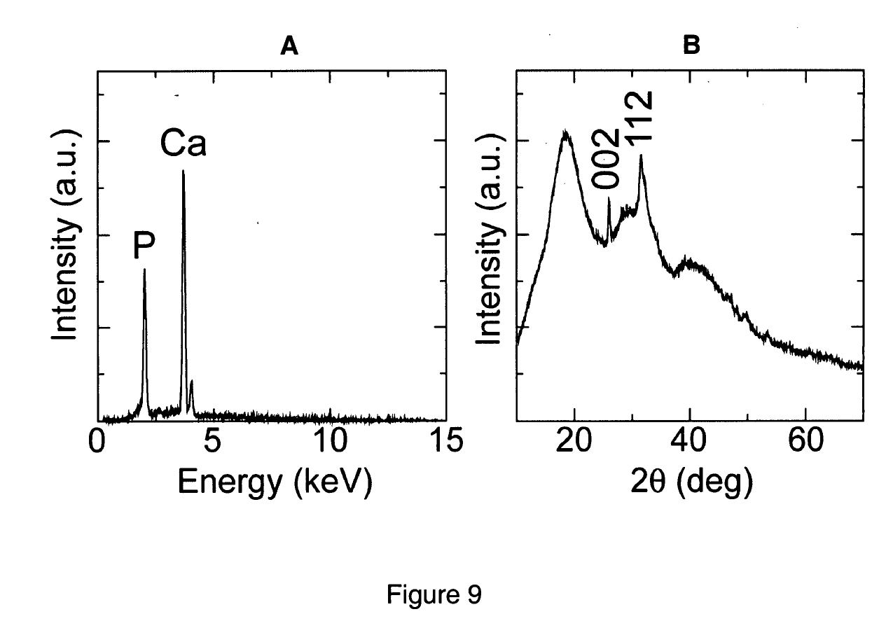

Referring to FIG. 9, EDS analysis performed on the spherical apatite aggregates (FIG. 9A) and XRD performed on the composite material produced at a heating rate of 0.2° C./min (FIG. 9B) confirmed the expected Ca/P ratio and typical reflections, (002) and (112), for crystalline HA (Saiz, E., et al., Biomaterials, 2002, 23, 3749-3756). The foregoing results suggest that a lower heating rate and a more sufficient overall mineralization time promote the formation of better-merged CP layers on the gel surface and that once the gel surface is fully covered with the initial nanocrystalline or amorphous CP layer, the growth of CP composed of large platelet crystallites that are easily detectable by XRD becomes energetically favorable.

The slowest linear heating rate attempted for the current investigation was 0.1° C./min (from room temperature to 95° C.). The most pronounced feature resulted from this mineralization condition was the dramatic increase of the number of nucleation sites formed on the pHEMA hydrogel surface. The longer exposure of the pHEMA gel to any given pH during the urea-mediated process is likely to lead to a more sufficient hydrolysis of the ethyl ester side chains, resulting in increased numbers of surface carboxylates that could serve as tight calcium ion binders and initial nucleation sites. The overwhelming amount of nucleation sites also made the distinction of 2-dimensional outward growth of circular CP layers difficult. A high-resolution image of some separate nucleation sites before they merge with each other, however, unequivocally showed the 2-dimensional outward growth pattern around these nucleation centers. X-ray elemental mapping of Ca and P again showed that the observed mineral patterns were composed of uniform calcium phosphate. EDS area analysis performed on the mineral surface revealed a Ca/P ratio matching that of HA. XRD of the composite showed typical reflections matching with those of crystalline calcium apatites. This is likely to result from the formation of crystalline aggregates of plate-like HA that were found over some nucleation sites within areas fully covered with amorphous CP layers.

### EXAMPLE 7

### Effect of Agitation of the Mineralization Solution in the Urea-Mediated Process

The influence of the agitation of the HA-urea mineral stock solution on the nucleation and precipitation behavior of the mineral was investigated using the same range of heating rates described above in Example 6. Upon constant stirring of the mineralization solution, HA was found to homogeneously precipitate out of the mineral solution with the gradual increase of pH. No nucleation and high-affinity growth of calcium apatites on the pHEMA gel surface was observed with any tested heating rates under constant agitation. This suggests that direct stirring of the HA-urea mineral stock solution interferes with the desired heterogeneous nucleation and growth of CP on the in situ generated acidic gel surface. Instead, it promotes homogeneous precipitation of HA across the solution, resulting in the formation of large amount of HA precipitates.

### EXAMPLE 8

### Composites made with HEMA Monomers and Co-Monomers having Various Anionic Functionalities

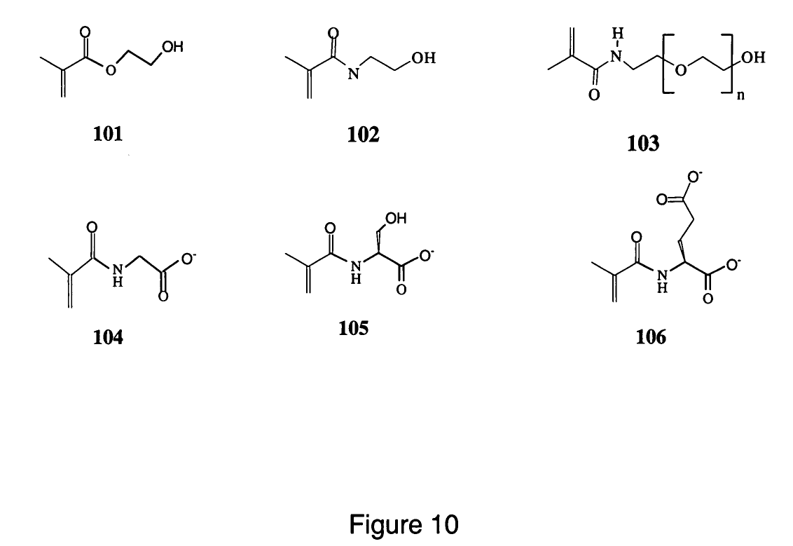

Hydrogels were derived from HEMA copolymerized with 5% each of three types of methacrylamide (MAm) monomers, each bearing one of the following anionic groups: glutamic acid (Glu), glycine (Gly) and serine (Ser). Referring now to FIG. 10, shown are monomers HEMA (101) and HEMAm (102), a co-monomer having a non-fouling residue (103), and co-monomers glycine-methacrylamide (GlyMAm, 104), serine-methacrylamide (SerMAm, 105) and glutamic acid-methacrylamide (GluMAm, 106). Co-monomers 104, 105, and 106 were synthesized by reaction of glycine, serine and glutamic acid with methacryloylchloride, respectively.

Other methacrylamide monomers were also designed and synthesized for further fine-tuning hydrogel copolymers' physical property, chemical versatility and biocompatibility but not used to make this hydrogel. For instance, HEMAm and monomers such as 103 carrying various numbers of non-fouling ethylene glycol units were synthesized. The more extended ethylene glycol linker (where n can be 1 to 500,000 but preferably from 1 to 1000) can be functionalized with terminal hydrazine or an aminooxy group for further elaboration of the hydrogel. For instance, chemoselective ligation of these terminal functionalities with aldehyde or ketone-bearing peptides, carbohydrate ligands or even metabolically engineered cells can be made to enhance biocompatibility of the hydrogel.

When the urea-mediated mineralization technique was applied to the mineralization of pHEMA-co-5%-pGluMAm, pHEMA-co-5%-pGlyMAm and pHEMA-co-5%-pSerMAm gels made according to the protocol in Example 1, similar mineralization patterns were observed as in Example 3. Two-dimensional circular calcium phosphate outward growth occurred from the nucleation sites. After a prolonged mineralization of 12 h, an amorphous or nanocrystalline calcium phosphate layer completely covered the gel surface, and on top of it, spherical aggregates of crystalline apatite precipitates formed as in the case of pHEMA in Example 3. During the deliberate fracturing of the composite did not lead to delamination of any circular mineral domains, suggesting an excellent gel-mineral interfacial adhesion strength.

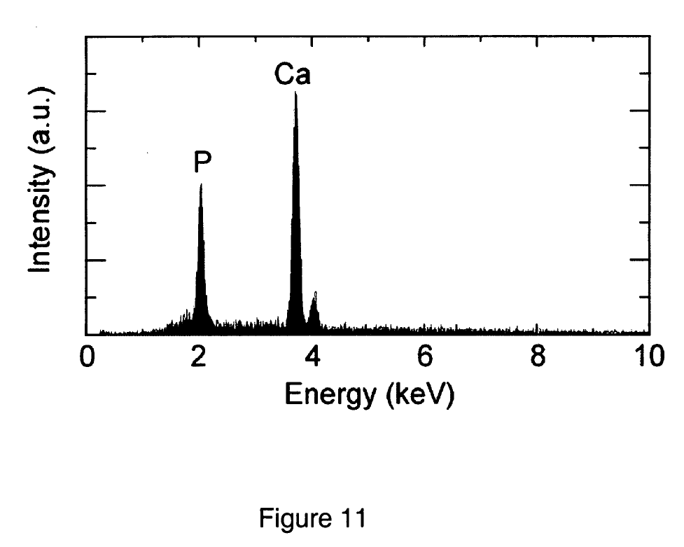

The EDS analysis (FIG. 11) of the mineral layer formed on the surface of mineralized pHEMA-co-5%-pGluMAm gel using the urea-mediated procedure showed two distinct peaks that correlate to Ca and P showing that the initial mineral layer of the composite is a thin calcium phosphate layer. Similar results were observed with mineralized pHEMA-co-5%-pGlyMAm and pHEMA-co-5%-pSerMAm gels.

The present examples, methods, procedures, specific compounds and molecules are meant to exemplify and illustrate the invention and should in no way be seen as limiting the scope of the invention. Any patents or publications mentioned in this specification are indicative of levels of those skilled in the art to which the patent pertains and are hereby incorporated by reference to the same extent as if each was specifically and individually incorporated by reference.

1. A bonelike composite, comprising:
a. a hydrogel polymer scaffold,
wherein said polymer comprises a polymerized compound, —CH₂—CR²—COOR¹)ₙ— wherein R¹ is H or lower alkyl, R² is H or a lower alkyl having from 1-20 carbon atoms and n is 10 to 100,000;
b. a mineral deposit on the surface and the interior of the hydrogel polymer, said mineral deposit bound by ionic charges between calcium ions and polymer groups remaining after hydrolytic cleavage of R¹, said mineral layer forming a nanocrystalline layer.

2. The composite of claim 1, wherein the hydrogel polymer scaffold has a water content between 20% and 100%.

3. The composite of claim 2, wherein said polymerized compound is pHEMA.

4. The composite of claim 2, wherein said polymer scaffold further comprising 0.1% to 50% a crosslinker.

5. The composite of claim 4, wherein said crosslinker is a compound of R³C(CH₂)—C(O)—X—R⁴—X—C(O)-C(CH₂)R^{3′}, wherein R³ and R^{3′} can be identical or different and can be H or a lower alkyl, wherein the number of alkyl groups is less than 10; R⁴ is an alkyl chain, [—(CH₂)ₙ—Y—(CH₂)_{n′}—]ₘ, wherein n and n′ are independently from 1 to 10, wherein m=1 to 500,000, X is O, S or N and Y is absent or O, S or NH.

6. The composite of claim 5, wherein said crosslinker is selected from the group consisting of diacrylates, diacrylamides, dimethacrylates or dimethacrylamides.

7. The composite of claim 6, wherein said crosslinker has a length varied from 1 to 500,000 repeating units.

8. The composite of claim 7, wherein said crosslinker bears a functional group which is selected from the group consisting of anionic groups, heteroatoms, polar ligands, aldehydes, ketones, phosphates, nucleic acids, amino acids, modified amino acids, glycosylated amino acids, phosphorylated amino acids, sulfated amino acids, peptides, proteins, carbohydrates, sugars, collagens, laminins, extracellular matrix components, biodegradable motifs and polyethylene glycols.

9. The composite of claim 8, wherein said crosslinker is ethylene glycol dimethacrylate, ethylene glycol dimethacrylamide or compound of CH₃C(CH₂)—CO—O—CH₂CH₃—O—CO—(CH₂)CCH₃ or CH₃C(CH₂)—CO—N—CH₂CH₃—N—CO—(CH₂)CCH₃.

10. The composite of claim 1, wherein said polymer scaffold further comprising 0.1% to 50% a co-monomer.

11. The composite of claim 10, wherein said co-monomer is a methacrylate or a methacrylamide.

12. The composite of claim 11, wherein said co-monomer bears a functional group which is selected from the group consisting of anionic groups, polar ligands, aldehydes, ketones, phosphates, nucleic acids, amino acids, modified amino acids, glycosylated amino acids, phosphorylated amino acids, sulfated amino acids, peptides, proteins, carbohydrates, collagens, laminins, extracellular matrix components, biodegradable motifs and polyethylene glycols.

13. The composite of claim 1, wherein said mineralization mixture is comprised of inorganic components selected from the group consisting of Ca²⁺, PO₄³⁻, OH⁻, CO₃²⁻, Cl⁻ and other trace inorganic elements.

14. The composite of claim 13, wherein the ratio of Ca²⁺ to PO₄³⁻ ions is between 0.5 and 4.

15. The composite of claim 14, wherein the ratio of Ca²⁺ to PO₄³⁻ ions is between 1 and 2.

16. The composite of claim 15, wherein said mineralization mixture is selected from the group consisting of crystalline, nanocrystalline or amorphous hydroxyapatite (Ca₁₀(PO₄)₆(OH)₂), calcium carbonate, dicalcium phosphate, tricalcium phosphate, octacalcium phosphate, calcium phosphates having a stoichiometry that ranges from CaO-2P₂O₅ to 4CaO—P₂O₅ and solubility behavior, under acidic and basic conditions, similar to that of hydroxyapatite.

17. The composite of claim 13, wherein the hydrolysis of R¹ is mediated by contacting the hydrogel polymer with a solution comprising said mineralization mixture and a mild base capable of modulating a slow increase in pH.

18. The composite of claim 17, wherein said initial mineral deposition is a nanocrystalline or amorphous mineral deposit.

19. The composite of claim 18, wherein said extended mineral layer is about 1 to 7 μm in thickness.

20. The composite of claim 19, attached to a bone in a vertebrate subject, or deposited upon an implant, or deposited upon organic-inorganic hybrid materials.

21. A composite structure prepared from:
a base monomer, —(CH₂—CR²—COOR¹)ₙ—, wherein R¹ is selected from the group consisting of H or lower alkyl; R² is selected from the group consisting of H or lower alkyl; and n is 10 to 100,000;
a cross linking agent selected from the group consisting of diacrylates, diacrylamides, methacrylates and methacrylamides; and
a mineralization mixture of calcium and phosphate, wherein said calcium and phosphate are in a ratio of 1Ca to 2P.

22. A method for preparing a bonelike composite, comprising:
a. forming a crosslinked hydrogel polymer, having a surface and an interior, comprised of a polymerized base monomer having ester-containing side chains,
b. hydrolyzing a percentage of the ester side chains to form reactive acidic groups on the surface and the interior of the hydrogel; and
c. contacting said reactive acidic groups with a mineral to form a nanocrystalline or amorphous mineral deposit on said acidic surface and interior of the hydrogel.

23. The method according to claim 22, wherein said base monomer has a structure, —CH₂—CR²—COOR¹)ₙ—, wherein R¹ is a lower alkyl group, wherein R₂ can be H or any lower alkyl group, wherein n is 10 to 100,000.

24. The method according to claim 23, wherein said base monomer is 2-hydroxyethyl methacrylate.

25. The method according to claim 22, wherein said crosslinking is 0.1% to 50%.

26. The method according to claim 25, wherein said crosslinker has a structure, R³C(CH₂)—C(O)—X—R⁴—X—C(O)—(CH₂)CR³, wherein R³ and R^{3′} can be H or a lower alkyl, wherein the number of alkyl groups is less than 10; R⁴ is [—(CH₂)ₙ—Y—(CH₂)_{n′—}]ₘ, wherein n and n′ are independently from 1 to 10, wherein m=1 to 500,000, wherein each R⁴ alkyl group can be independently the same or different; wherein X is O, S or N and Y is absent or O, S or NH.

27. The method according to claim 26, wherein said crosslinker is selected from the group consisting of dimethacrylate, dimethacrylamide, di acrylate or diacrylamide.

28. The method according to claim 27, wherein said crosslinker is ethylene glycol dimethacrylate, ethylene glycol dimethacrylamide or a compound of R³C(CH₂)—C(O)—X—R⁴—X—C(O)—(CH₂)CR³, wherein R³ and R^{3′} are both CH₃, R⁴ is CH₂CH₃ and X is O.

29. The method according to claim 22, wherein said polymer further comprises 0.1% to 50% a co-monomer.

30. The method according to claim 29, wherein said co-monomer is a methacrylate or a methacrylamide.

31. The method according to claim 30, wherein said co-monomer bears a functional group which is selected from the group consisting of anionic groups, polar ligands, aldehydes, ketones, phosphates, nucleic acids, amino acids, modified amino acids, phosphorylated amino acids, glycosylated amino acids, sulfated amino acids, peptides, proteins, carbohydrates, sugars, collagens, laminins, extracellular matrix components, biodegradable motifs and polyethylene glycols.

32. The method of claim 22, wherein said hydrolysis of R¹ groups is catalyzed by the gradual addition or in situ generation of an acid, base or an esterase enzyme that will thermally or aqueously degrade to release acid or base in a mild fashion in the interior and on the surface of the hydrogel.

33. The method of claim 32, wherein said base is selected from the group consisting of ammonia, ammonium hydroxide, urea, piperidine, imidazole, potassium carbonate, sodium carbonate, potassium bicarbonate, sodium bicarbonate,and pyridine.

34. The method of claim 33, wherein said acid is selected from the group consisting of hydrochloric acid, sulfuric acid, phosphoric acid, acetic acid, formic acid, citric acid, carboxylic acid, and organic acids miscible with water.

35. The method according to claim 34, wherein said hydrolysis of R¹ groups is caused by gradual heating of urea in water.

36. The method according to claim 35, wherein said gradual heating of urea is from room temperature to 95° C. at a heating rate between 0.1° C./min and 1° C./min.

37. The method according to claim 36, wherein said heating rate is a constant heating rate between 0.2 and 0.5° C./min.

38. The method according to claim 37, wherein said heating of urea is without agitation or stirring.

39. The method according to claim 22, wherein said mineral deposited into and on the surface of the hydrogel is a calcium phosphate.

40. The method according to claim 39, wherein the ratio of calcium to phosphate in said mineral is between 0.5 and 4.

41. The method according to claim 40, wherein said mineral is nanocrystalline or amorphous hydroxyapatite (Ca₁₀(PO₄)₆(OH)₂).

42. A method for preparing a bonelike composite, comprising:
a. contacting a hydrogel scaffold with a solution comprised of urea and a mineral, wherein said hydrogel scaffold is comprised of a crosslinker and a monomer, wherein said monomer is a substituted or unsubstituted polyacrylate derivative having ester-containing side chains and said crosslinker is selected from the group consisting of dimethacrylate, dimethacrylamide, diacrylate or diacrylamide;
b. heating said solution to decompose urea and increase pH to hydrolyze said ester-containing side chains to form reactive acidic groups on the surface and in the interior of the hydrogel scaffold; and
c. forming a mineral deposit on the surface and interior of the hydrogel, wherein mineral deposition occurs as a result of nucleation at the acidic groups on the surface and interior of the hydrogel.

43. The method of claim 42, wherein said mineral is selected from the group consisting of: hydroxyapatite, calcium carbonate, calcium phosphates, dicalcium phosphate, tricalcium phosphate, and octacalcium phosphate.

44. The method of claim 43, wherein the ratio of calcium to phosphate in the mineral deposit is between 0.5 and 4, preferably from 1 to 2, wherein the calcium phosphate stoichiometry can range from CaO-2P₂O₅ to 4CaO—P₂O₅.

45. The method of claim 44, wherein the hydrogel is formed from HEMA monomer and a crosslinker selected from the group consisting of ethylene glycol dimethacrylate or ethylene glycol dimethacrylamide.

46. The method of claim 45, wherein the pH is increased from about 1-3 to about 7-9.

47. The method according to claim 46, wherein said gradual heating of said solution is from room temperature to 95° C. at a heating rate between 0.1° C./min and 1° C./min.

48. The method according to claim 47, wherein said heating rate is a constant heating rate between 0.2 and 0.5° C./min.

49. The method according to claim 47, wherein said heating of solution is without agitation or stirring.

50. The method according to claim 49, wherein said heating of said solution is extended to about 10 to 12 hours to form an extended mineral layer upon said mineral deposit.
